(19) United States
(12) Patent Application Publication
(10) Pub. No.: US 2021/0030998 A1
(43) Pub. Date: Feb. 4, 2021

(54) APPARATUS FOR IMPROVING MENTAL WELL BEING, AND A METHOD THEREOF

(71) Applicant: Well Being Digital Limited, Pak Shek Kok (HK)

(72) Inventor: Ming Yip Wallace WONG, Pak Shek Kok (HK)

(21) Appl. No.: 16/921,599

(22) Filed: Jul. 6, 2020

(30) Foreign Application Priority Data
Aug. 4, 2019 (HK) 19127712.8

(51) Int. Cl. A61M 21/02; A61B 5/024; A61N 5/06; H04R 1/10

(52) U.S. Cl. CPC A61M 21/02; A61B 5/02433; A61M 2021/0033; H04R 1/1016; A61N 5/0618

(57) ABSTRACT

An earpiece and a method of for improving mental well-being in a person, using flashing light suppled into the ear or ears of a person. The earpiece can be worn on one ear instead of two for inducing a binaural beats effect.

### CROSS-REFERENCE TO RELATED APPLICATIONS

This application claims priority to Hong Kong patent application no. 19127712.8 filed in the Hong Kong patent office on Aug. 4, 2019 and entitled, “AN APPARATUS FOR IMPROVING MENTAL WELL BEING”, which is incorporated herein by reference in its entirety.

### FIELD OF INVENTION

The invention relates to the field of binaural beats devices.

### BACKGROUND OF THE INVENTION

It is believed that listening to low frequency sounds helps people to sleep and relax. Furthermore, it appears that low frequency sounds are useful for alleviating psychological, emotional and even some physical problems. Typically, these sounds have to be at a frequency of 40 Hz or below. Unfortunately, such low frequency is below the audible range of the human ear. Hence, binaural beats have been used to create in a person the effects of hearing a low frequency sound.

A low frequency sound produced using binaural beats is actually an auditory illusion. The method comprises supplying pure tone sounds of two different frequencies to a person. The sound of one frequency is supplied to one ear and the sound of the other frequency is supplied to his other ear, causing the person to hear dichotic sounds. This creates an auditory illusion of hearing a tone of a frequency which is the difference between the frequencies of the two supplied sounds.

Typically, the two frequencies are below 1500 Hz and the difference between the two frequencies is 40 Hz or less. Therefore, if a pure tone of 530 Hz is supplied to the right ear and a pure tone of 520 Hz is supplied to the left ear, the dissonance of the two frequencies in his ears causes a person to hear an illusory tone of 10 Hz.

Binaural beats providing an illusory tone of 8 Hz to 10 Hz have been associated with improved learning, memorisation and creativity in the listener. On the other hand, binaural beats providing an illusory tone of 10 Hz to 14 Hz have been associated with inducing in the listener enhanced serotonin release, mood elevation, better clarity, increased focus and increased awareness. Typically, auditory illusion of less than 8 Hz is difficult to create.

Supplying each ear with a pure tone sound requires a person to wear earphones on both his ears. This requires that the earphones dominate the person's aural sense completely, which makes it impossible for the person to listen to binaural beats while engaging in activities which require hearing, such as listening to instruction during yoga classes.

Accordingly, it is desirable to provide an apparatus and a method for a person to enjoy the therapeutic benefits of binaural beats without needing his aural sense to be completely dominated for the sole purpose of creating the auditory illusion, so that there could be a possibility using his hearing for other activities at the same time.

### STATEMENT OF INVENTION

In a first aspect, the invention proposes an apparatus for improving mental well-being comprising: at least one earpiece; the at least one earpiece having at least one light source arranged to emit flashing light into the canal of an ear wearing the earpiece.

Depending on the flashing frequency, the mind can be conditioned so as to relax the mind, put the mind at rest or into a meditative state, or to improve mental concentration, short term or long term memory, or enhance quickness of the mind, deepen sleep, or the heighten the ability of the mind to make analyses and so on. When these mental capacities are improved, it could affect positively a person's effectiveness in his daily tasks, his joviality and conviviality, and impart a general sense of well-being.

The invention provides the possibility of obviating the need to supply sounds of different frequencies to both ears of a person to create the same auditory illusion produced by binaural beats. Instead, a single earpiece comprising a flashing light source is sufficient to create the same or similar neural response. Therefore, only one ear may need to wear an earpiece, instead of both ears.

Typically, the apparatus is wearable on the ear of a user, as this allows the therapeutic effect to arise slowly over time.

The invention also provides the possibility that the person's sense of hearing is not occupied by sounds for creating binaural beats effects. Hence, the ears are free for aural deployment, to listen to music or to allow the person to engage in conversation, all while the person is receiving therapy which is the same or similar to that brought about by binaural beats effects. This feature could open a new possibility for people to be in group therapy using binaural beats, such as meditation or yoga classes in which participants are aided by binaural beats effects and are not prevented from hearing instruction.

It has been impossible for the deaf to enjoy the benefits of binaural beats, as the sound-based prior art relies on a person being able to hear in both ears. In contrast, the invention does not require the user to be capable of hearing; a deaf person who is responsive to light-based stimulation of his brain via one of his ear canals can enjoy the therapeutic effects of binaural beats.

Preferably, the light source emits light in a periodic flashing frequency. Typically, the flashing frequency of the light source is 40 Hz or below. Advantageously, however, the flashing frequency of the light source is lower than 8 Hz, such as 6 Hz, which possibly induces better relaxation in a person. Sound-based binaural beats prior art tends to create an auditory illusion of a tone that may be as low as only 8 Hz but not lower, possibly because the brain finds it harder to trick itself into hearing an illusory tone of less than 8 Hz. The present invention is unlikely to face the same limitation, as it does not require deceiving the brain and may therefore go below 8 Hz.

Preferably, the apparatus is capable of adjusting the flashing frequency in response to physiological feedback. For example, the apparatus comprises a heartbeat monitor for obtaining physiological feedback from heartbeat. Typically, the heartbeat monitor is an electrocardiogram sensor.

An electrocardiogram sensor does not use light-based measurement, and the electrodes of which may therefore be integrated into the apparatus and inserted into the ear canal of the person without affecting the binaural beats effects produced by the light source.

Alternatively, the heartbeat monitor is a photoplethysmography sensor, in which case the photoplethysmography sensor is preferably worn on a part of the person's body, such as the wrist, and is not normally configured to be placed into the ear canal of the person.

Preferably, the at least one earpiece has a further light source arranged to emit light into the same ear canal; and the one light source and the further light source are each capable of emitting light in different electromagnetic frequencies; and the one light source and the further light source are capable of emitting light alternatively.

Optionally, the apparatus further comprises another earpiece having a light source arranged to emit light into the canal of the other ear; the light sources of the two earpieces capable of emitting in the same flashing frequency.

Preferably, the light sources of the earpieces emit in the same phase.

Preferably, the apparatus further comprises another earpiece; wherein the earpiece and the other earpiece are capable of supplying sound to generate an auditory illusion for improving mental well-being. The use of two earpieces to emit flashing light into both ear canals intensifies the user's binaural beats experience. Typically, the light sources of the two earpieces emit light at the same flashing frequency. Furthermore, it is preferable that the light sources in the earpieces emit at the same flashing frequency and in the same phase, so that the binaural beats effects which the person experiences further intensifies.

In a further aspect, the invention proposes a method of improving mental well-being comprising a step of emitting flashing light into at least one ear canal.

Typically, the method further comprises a step of: emitting flashing light into in a periodic flashing frequency. In use, the method is typically applied over relatively long period of time, such as 5 minutes, an hour or even more.

Optionally, the flashing frequency of the flashing light is 6 Hz or below.

Typically, the method further comprises a step of: emitting another flashing light into the ear canal of the person; wherein the flashing light and the other flashing light each emits alternatively; and the flashing light and the other flashing light each has a different electromagnetic frequency.

Preferably, the method further comprises a step of: flashing light into the other ear canal of the person.

Preferably, the method further comprises a step of: synchronizing the flashing frequencies in both ear canals, such that the flashings are in phase.

Optionally, the method further comprises a step of: providing sound into the ears to generate an auditory illusion for improving mental well-being. In other words, a person can enjoy the therapeutic effects of both sound-based and light-generated binaural beats effects at the same time. This possibly provides enhanced therapeutic benefits. Preferably, the auditory illusion created has a frequency which is the same as the flashing frequency of the flashing light. Alternatively, the method comprises a step of providing comprehensible sounds into the ears, such as music. This provides the possibility of the person enjoying the benefit of binaural beats while listening to music or an audio book, or engaging in a tele-conversation.

Advantageously, the apparatus and method are not limited to being used on a person but may be used on any animal. An anxious race horse can be calmed by using the method since a light based relaxation apparatus is unlikely to alarm the aural sense of the animal.

### BRIEF DESCRIPTION OF THE FIGURES

It will be convenient to further describe the present invention with respect to the accompanying drawings that illustrate possible arrangements of the invention, in which like integers refer to like parts. Other arrangements of the invention are possible, and consequently the particularity of the accompanying drawings is not to be understood as superseding the generality of the preceding description of the invention.

FIG. 1 shows an embodiment of the invention;

FIG. 2 shows a variation to the embodiment of FIG. 1;

FIG. 3 shows a variation to the embodiment of FIG. 1;

FIG. 4 shows the embodiment of FIG. 1 connected to a smartphone;

FIG. 5 is a schematic diagram of some of the components in the embodiment of FIG. 1;

FIG. 6 shows ECG pulses read by an ECG sensor installed in an embodiment which is a variation of the embodiment of FIG. 1;

FIG. 7 shows a variation to the embodiment of FIG. 1;

FIG. 8 shows a variation to the schematic embodiment of FIG. 5;

FIG. 9 shows the embodiment of FIG. 1 used on both a person's ears;

FIG. 10 shows the flashing of light provided by the embodiment of FIG. 9; and

FIG. 11 shows a variation of the embodiment of FIG. 1.

### DETAILED DESCRIPTION OF EMBODIMENTS

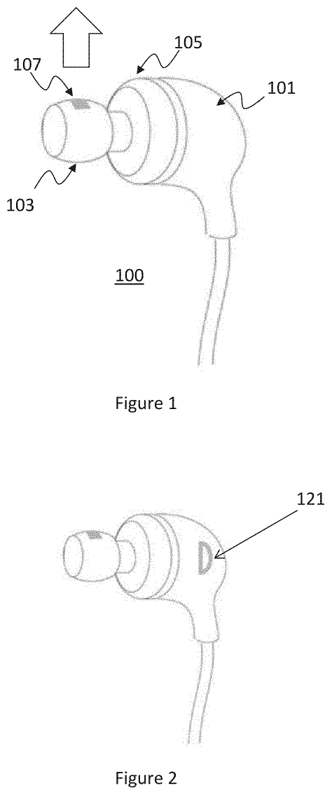

FIG. 1 shows an embodiment which is an earpiece 100. The earpiece 100 comprises a housing 101 from which a nozzle 103 extends. The nozzle 103 can be inserted into an ear hole to be situated inside the ear canal.

The perimeter of the housing 101 is padded with a resilient, deformable material 105. When the nozzle 103 is pushed into an earhole, the resilient, deformable material deforms to fill the ear hole and fits snugly therein. Ambient light and sound is blocked from entering the earhole by this resilient, deformable material.

The nozzle 103 is provided with at least one light source 107. Typically, the light source 107 is an LED (light emitting diode). In one embodiment, the light source 107 is arranged along the side of the nozzle 103 and emits light somewhat radially of the nozzle 103 as illustrated by the white arrow in FIG. 1 such that, if the earpiece is worn on a person's ear, the emitted light passes through the wall of the ear canal. Light that has penetrated the ear canal wall is scattered therein by tissue and blood, and a portion of the light is deflected towards the inner parts of the head.

It has been found that light shining into the ear canal stimulates a response in the brain (Starck et. al. [2012] Stimulating brain tissue with bright light alters functional connectivity in brain at the resting state, World Journal of Neuroscience, 2, 81-90). The exact mechanism underlying this neurological reaction to light is still uncertain. A couple of explanations have been proposed. Firstly, phototransduction could be provided by panopsin, a protein found in the brain. Panopsin belongs to a group of protein called opsins which are light sensitive. This kind of proteins is also found in the photoreceptor cells of the retina. Secondly, there could be a phototransduction mechanism via neurotransmitters like dopamine, serotonin or noradrenaline.

Whatever the underlying mechanism may be, the phenomenon observed by Starck is exploited in the embodiment to create in a person an experience of binaural beats, as if he is listening to a pure-tone sound of a very low frequency but without actually listening to anything. The frequency of the flashing of light in the ear canal causes the brain to produce responses which give the person a neurological experience that is the same as or similar to that produced when the person's brain is tricked by the prior art into hearing a tonal illusion of the same frequency. Advantageously, unlike the prior art, the embodiment does not require the person to listen to pure-tone sounds using both ears; supplying a light source to only one ear is sufficient to produce a binaural beats effect.

Therefore, in the simplest embodiments, this leaves at least one ear free from having to wear a device.

As a flashing light source 107 is used to create a binaural beats effect, and no hearing is required, the term ‘binaural’ is somewhat a misnomer. This term is retained in use herein, nevertheless, for ease of description.

By ‘flashing’, the meaning here is akin to ‘blinking’. The skilled reader would understand that ‘flashing frequency’ does not mean the electromagnetic frequency of the light emitted by the light source 107, but the number of times the light source 107 is switched ON and OFF in a unit of time.

Typically, the light source 107 is switched ON and OFF completely in periodic cycles to create the flashing. Alternatively, only the intensity of the light changes, that is, the intensity of the light is merely increased and decreased in periodic cycles, or brightened and dimmed, without going so far as to being switched OFF completely. The periodic fluctuation between brightening and dimming of light creates the same effect that created by continually and successively switching the light completely ON and OFF.

The preferred flashing frequency is 6 Hz, which is believed to induce better relaxation than the relatively higher frequency. Sound-based binaural beats prior art tends to create an auditory illusion of a tone that may be as low as only 8 Hz but not lower, possibly because the brain finds it harder to trick itself into hearing an illusory tone of less than 8 Hz. The embodiment is unlikely to face the same limitation, as it does not require deceiving the brain and may therefore go below 8 Hz.

Optionally, the flashing frequency is pre-determined at factory, and the person using the embodiment has no means of changing the flashing frequency. In this case, the embodiment is specifically manufactured for providing only one type of binaural beats therapeutic effect for the user.

Preferably, however, the embodiment further comprises a control mechanism for the person to select the flashing frequency. An adjustment dial 121 can be provided on the earpiece, as shown in FIG. 2, and turning the adjustment dial 121 changes the flashing frequency. As typical of such dials, there are numbers (not illustrated) on the dial to indicate against a marker the selected flashing frequency. An adjustment dial is more suitable for controlling the light source 107 where the light source 107 is part of an analogue circuit.

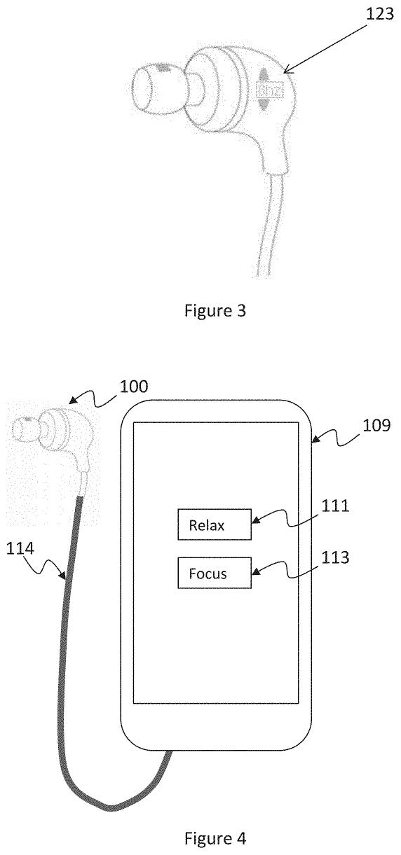

Alternatively, as shown in FIG. 3, a digital control and display 123 is provided on the earpiece for select the flashing frequency of the light source 107. The person using the embodiment just presses the UP and DOWN buttons to select the flashing frequency. A digital display tends to be more suited to a digital control system, in which case the earpiece would comprise a microprocessor, and any necessary memory and firmware, for operation.

More preferably, however, control of the flashing frequency is provided in the form of software application, or an ‘app’ installed in a smartphone 109, as illustrated in FIG. 4. The smartphone is in communication with the earpiece 100 in order to allow the app to control the operation of the earpiece 100. The app allows the person to specify the flashing frequency to use, such as by selecting the flashing frequency on a frequency slide bar (not illustrated). Alternatively, the app presents a graphical user interface which displays therapeutic binaural beats benefits which the person could select. The person interacts with the graphical user interface to select the desired benefits, i.e. the person selects the graphical button for “Relax” 111 or “Focus” 113 in the app.

When the person has made his selection, the app automatically looks up in pre-stored information the flashing frequency required for providing the selected therapeutic benefit, and then controls the light source 107 in the earpiece to flash in that flashing frequency. The pre-stored information is typically stored in the smartphone as part of the app.

The two choices of Relax 111 and Focus 113 shown in FIG. 4 are only examples. In an actual product comprising an embodiment, many more benefit options which can be provided by different flashing frequencies can be presented as options.

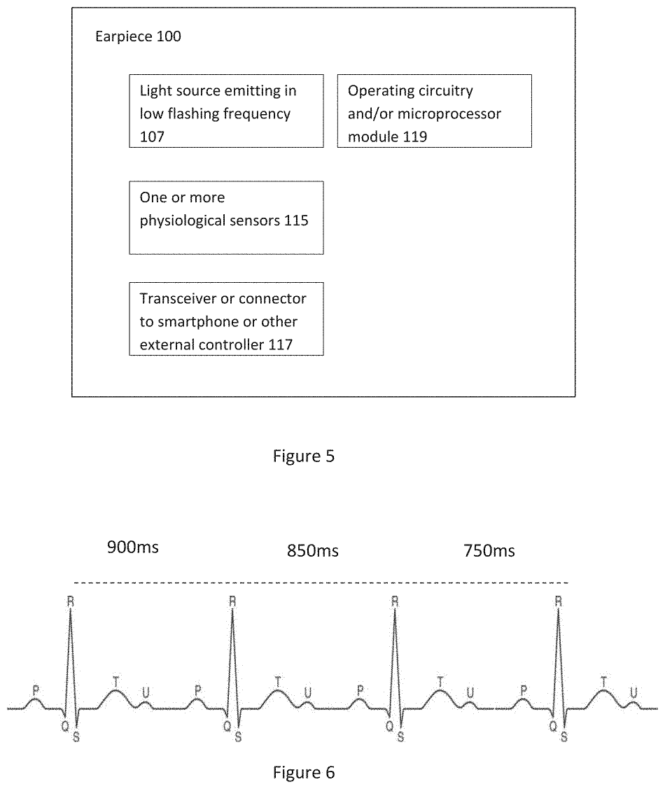

FIG. 5 shows a schematic diagram of possible components in the embodiment of FIG. 1. The schematic diagram illustrates the earpiece 100 comprising the light source 107, and an operating circuit or a microprocessor module 119 to operate the light source 107, and to cause the light source 107 to flash at a selected flashing frequency.

Furthermore, FIG. 5 shows that the earpiece 100 comprises a connector or transceiver 117 for providing communicating between the microprocessor module 119 and an external device, such as the smartphone 109 aforementioned. Preferably, the communication is wireless and the transceiver communicates with the smartphone 109 through Wi-Fi, Bluetooth or other known wireless communication technology. Alternatively, communication is provided by a physical cable, in which case the earpiece has the aforementioned connector to be connected with a cable linked to the external device. In relation to this, FIG. 4 illustrates the earpiece 100 connected to a smartphone 109 by cable 114.

Preferably, the earpiece further comprises physiological sensing components 115 for monitoring whether a person using the embodiment has any adverse reaction to the flashing light. For example, the earpiece further comprises an electrocardiogram (ECG) sensor. The person's physiology, such as his heartrate or intervals between his ECG pulses, can be monitored. If any adverse reaction is observed, such as an uncharacteristic increase in heartbeat, the earpiece can alert the person by causing an alarm to be raised via the connected smartphone 109.

There are many ways of raising an alarm besides raising the alarm via the smartphone 109. These include causing the earpiece to flash an alarm LED which is visible to people around the person.

Besides using an ECG sensor to monitor adverse reaction, in a variation of the embodiment, the flashing frequency may be determined by physiological data observed by the ECG sensor. For example, the ECG sensor is used to monitor the Heart Rate Variability (HRV) of the person.

As the skilled man knows, an ECG sensor measures electrical signals that control the expansion and contraction of heart chambers. A healthy heartbeat always exhibit a normal amount of irregularities. The pumping of the heart does not recur in strictly regular periods; the period of time between every two heartbeats is always varied. Using an ECG sensor, the variation of the interval between heartbeats can be measured. This is illustrated in FIG. 6, which shows three intervals among four heartbeats, all of which are different. The extent of variation can be quantified by known statistical methods, such as rMSSD (Root Mean Square of the Successive Differences).

If the heartbeat is very regular and the period between each beat is relatively consistent, it means the HRV is low. A low HRV indicates that the person is highly stressed. In the present embodiment, if the HRV obtained by ECG data shows that the person is highly stressed, the app automatically selects a pre-determined flashing frequency for the light source 107 to create binaural beats effects that could help the person relax and calm down. For example, the lower the HRV, the higher the stress level, the lower the flashing frequency selected. The correlation of preferred flashing frequency to the stress level is a matter of implementation choice by different embodiment manufacturers.

Alternatively, the physiological sensing component 115 for monitoring the heartbeat of the person could be a PPG (photoplethysmography) sensor. A PPG sensor uses a light-based technology to sense the surges of arterial blood flow as controlled by the heart's pumping action. To avoid having an effect on the brain, the PPG sensor is worn on another part of the body, such as the wrist, instead of being placed on the earpiece 100 to be inserted into the ear canal. The PPG sensor is preferably in wireless communication with the app in the smartphone. As with the ECG, HRV of the person observed from PPG pulses may be used by the app to adjust the flashing frequency of the light source 107 in the earpiece 100.

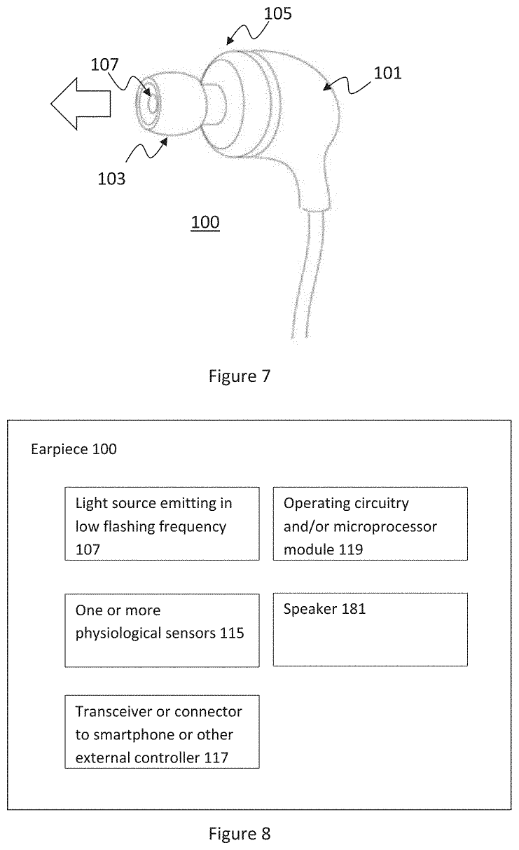

FIG. 7 shows a variation of the embodiment in which the light source 107 is placed at the tip of the nozzle 103 to shine directly towards the ear drum, i.e. in the direction illustrated by the white arrow. Shining light directly at the ear drum may give a better effect than shining light radially into the wall of the ear canal, as the blood and tissue components in and around the ear drum are more directly stimulated by the flashing, and may be better and more direct effects on the brain.

In the aforementioned embodiments, the earpiece 100 is not an earphone and does not need to have a speaker to supply sounds into the ears. Preferably, however, the earpiece 100 does comprise a speaker 181 which is able to provide sounds into the ear. See FIG. 8 illustrating this. This allows the earpiece 100 be used as an earphone or a hearing aid device if connected to a supply of sound signals. Advantageously, the earpiece 100 can then be used to provide music to the person when connected to a music playing device such as the smartphone 109 or iPod™, while the light source 107 is flashing into his ear canal to create a binaural experience. Furthermore, the person can even engage in a tele-conversation via the smartphone 109 while enjoying binaural beats therapy.

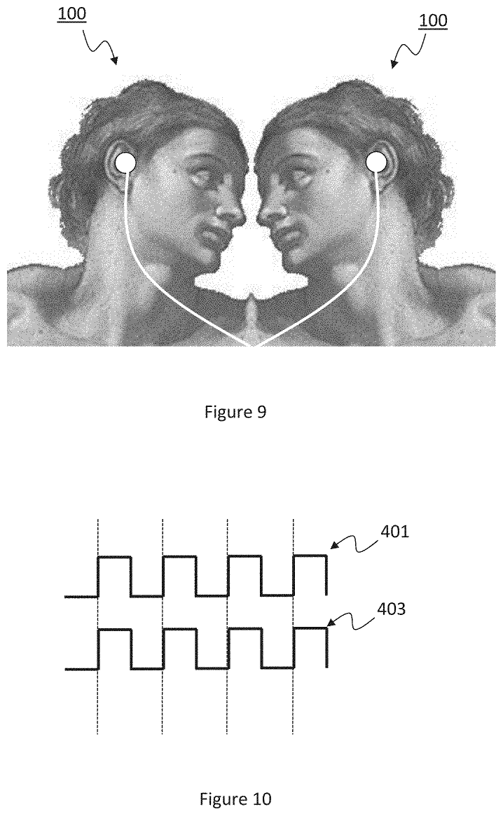

FIG. 9 illustrates another embodiment which comprises two earpieces. The person using the embodiment wears an earpiece 100 in each of his ears. Each of the earpieces has a speaker for playing a tonally pure sound in a different frequency, to create a sound-based binaural beat. There are at least two variations to such a two-piece configuration. Firstly, only one of the two earpieces comprises a light source for flashing light into an ear canal of the person. In a second variation, both of the earpieces comprise a light source each, for flashing light into both ear canals of the person.

The first variation of the embodiment of FIG. 9 is able to produce reinforced binaural beats effects, by using both sound-based binaural beats and light-based binaural beats at the same time. However, the light based binaural beats effects are produced by a flashing light source 107 in only one of his ears. The frequency of the illusory tone produced by the sound-based binaural beats is the same as the flashing frequency of the light source 107 in the ear, and the cycles are preferably synchronized and in phase. For example, if the tonally pure sound played by the speaker one earpiece 100 is at 1200 Hz, and the tonally pure sound played by the other earpiece is at 1160 Hz, thus generating a differential frequency of 40 Hz in binaural beats, then the light is also flashing at the frequency of 40 hz. In this way, the sound-based binaural beats and the flashing of the light source 107 provide together a reinforced binaural beat experience. This embodiment is useful if the person wishes to benefit from a binaural beat experience while he is working or travelling, and there are mind-drawing distractions in the surroundings which may be overcome by the reinforcement.

FIG. 10 shows two square waves, labelled 401 and 403. The top square wave 401 represents the low frequency illusory tone induced by sound-based binaural beats. The bottom square wave 403 represents flashing of light emitted by the light source 107 into one of the ear canals of the person. The light source 107 is flashing at the same frequency as the frequency of the illusory tone. Both the square waves are shown having the same frequency, and in phase.

In the second variation of the embodiment of FIG. 9, as explained, each of the two earpieces 100 comprises a light source 107. Preferably, both the light sources are able to emit flashing light into the respective ear canal in the same flashing frequency and phase. Furthermore, the frequency of the illusory tone is also the same and in phase.

To enable synchronization between the illusory binaural beats produced by pure tone sounds and the flashing light sources 107, there must be communication between the two earpieces 100. Preferably, the earpieces 100 use wireless transceivers to communication with each other, although physical connection between the two earpieces via a communication cable is also possible. Preferably, instead of being in communication with each other directly, both earpieces 100 are in communication through the smartphone 109.

Typically, the light source 107 emits light in monochromatic electromagnetic frequency, which in practice is usually a very narrow bandwidth about a target frequency. Certain electromagnetic frequencies are preferred, such as those of the red and infrared region, which are more capable of penetrating into human tissues than other electromagnetic frequency. However, light of any colour, such as red, green and blue may be used, or in the invisible electromagnetic frequencies, such as infrared.

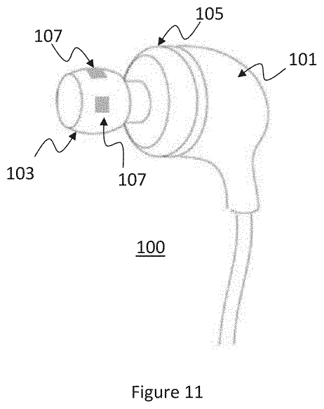

In yet another embodiment, as shown in FIG. 11, two light sources 107 (labelled with the same reference numbers) are provided on the nozzle of a single earpiece 100. Each light source 107 emits light in a different electromagnetic frequency. When in operation, the two light sources 107 are turn ON and OFF in succession one after the other, so that the flashing of the two light sources 107 do not overlap. The advantage of this embodiment is to stimulate different types of tissue and blood component in the ear canal by using two different electromagnetic frequencies, which reinforces the binaural beats effects created by the flashing light sources 107.

The combination of the electromagnetic frequencies can be varied. In one combination, both the light sources 107 emit visible light, albeit in different electromagnetic frequency. In another combination, each of the light sources 107 emits invisible light. In yet another combination, one of the light sources 107 emits invisible light while the other one of the light sources 107 emits visible light. For example, the two light sources 107 may emit light in red and blue respectively, or the two light sources 107 may emit light in near infrared and far infrared respectively, or the two light sources may emit light in blue and in infrared respectively.

Accordingly, the embodiments described includes a wearable apparatus 100 for improving mental well-being in a person comprising: at least one earpiece; the at least one earpiece having at least one light source 107 arranged to emit light into the canal of an ear wearing the earpiece; wherein the light source 107 is capable of emitting flashing light into the ear canal. Typically, the electromagnetic frequency of the light source 107 is one which is capable of inciting a neurological response, such as and not limited to infrared that can be absorbed by blood. The light source 107 flashes at a periodic flashing frequency, and the apparatus is capable of being worn over relatively long period of time. Hence, the person wearing the wearable apparatus 100 over an extended period of time will have a light flashing periodically into his ear canal all the time while he is wearing the apparatus 100.

Depending on the flashing frequency, the mind can be conditioned so as to relax the mind, put the mind at rest or into a meditative state, or to improve mental concentration, short term or long term memory, or enhance quickness of the mind, deepen sleep, or the heighten the ability of the mind to make analyses and so on. When these mental capacities are improved, it could affect positively a person's effectiveness in his daily tasks, his joviality and conviviality, and impart a general sense of well-being.

Hence, embodiments may be used by people who are depressed, people who feels listless or feels a sense of anger or annoyance to try and curb these emotions.

Furthermore, the embodiments described includes method for improving mental well-being in a person, comprising a step of emitting flashing light into at least one ear canal.

While there has been described in the foregoing description preferred embodiments of the present invention, it will be understood by those skilled in the technology concerned that many variations or modifications in details of design, construction or operation may be made without departing from the scope of the present invention as claimed.

For example, although flashing light having a periodic flashing frequency have been described, it is within contemplation that some embodiments comprises flashing light that flashes aperiodically, i.e. non-periodically, into an ear canal.

Furthermore, the embodiments are not limited to being used on people but any animal which has ears to wear an earpiece device, such as a racehorse.

1. An apparatus for improving mental well-being comprising:
at least one earpiece;
the at least one earpiece having at least one light source arranged to emit flashing light into the canal of an ear wearing the earpiece.

2. An apparatus for improving mental well-being as claimed in claim 1, wherein
the light source emits light in a periodic flashing frequency.

3. An apparatus for improving mental well-being as claimed in claim 2, wherein
the flashing frequency is 6 Hz.

4. An apparatus for improving mental well-being as claimed in claim 2, wherein
the apparatus is capable of adjusting the flashing frequency in response to physiological feedback.

5. An apparatus for improving mental well-being as claimed in claim 4, wherein
the apparatus comprises a heartbeat monitor for obtaining physiological feedback from heartbeat.

6. An apparatus for improving mental well-being as claimed in claim 5, wherein
the heartbeat monitor is an electrocardiogram sensor.

7. An apparatus for improving mental well-being as claimed in claim 5, wherein
the heartbeat monitor is a photoplethysmography sensor.

8. An apparatus for improving mental well-being as claimed in claim 1, wherein
the at least one earpiece has a further light source arranged to emit light into the same ear canal; and
the one light source and the further light source are each capable of emitting light in different electromagnetic frequencies; and
the one light source and the further light source are capable of emitting light alternatively.

9. An apparatus for improving mental well-being as claimed in claim 1, further comprising
another earpiece having a light source arranged to emit light into the canal of the other ear;
the light sources of the two earpieces capable of emitting in the same flashing frequency.

10. An apparatus for improving mental well-being as claimed in claim 9, wherein
the light sources of the earpieces emit in the same phase.

11. An apparatus for improving mental well-being as claimed in claim 1, further comprising
another earpiece; wherein
the earpiece and the other earpiece are capable of supplying sound to generate an auditory illusion for improving mental well-being.

12. A method of improving mental well-being comprising a step of:
emitting flashing light into at least one ear canal.

13. A method of improving mental well-being as claimed in claim 12, further comprising a step of:
emitting flashing light into in a periodic flashing frequency.

14. A method of improving mental well-being as claimed in claim 12, wherein
the flashing frequency of the flashing light is 6 Hz or below.

15. An method of improving mental well-being as claimed in claim 12, further comprising a step of:
emitting another flashing light into the ear canal of the person; wherein
the flashing light and the other flashing light each emits alternatively; and
the flashing light and the other flashing light each has a different electromagnetic frequency.

16. An method of improving mental well-being as claimed in claim 12, further comprising a step of:
flashing light into the other ear canal of the person.

17. An method of improving mental well-being as claimed in claim 16, further comprising a step of:
synchronizing the flashing frequencies in both ear canals, such that the flashings are in phase.

18. A method of improving mental well-being as claimed in claim 12, further comprising a step of:
providing sound into the ears to generate an auditory illusion for improving mental well-being.

19. A method of improving mental well-being as claimed in claim 18, wherein
the auditory illusion has a frequency which is the same as the flashing frequency of the flashing light.
